(19) United States
(12) Patent Application Publication
(10) Pub. No.: US 2023/0112384 A1
(43) Pub. Date: Apr. 13, 2023

(54) COMPOSITION COMPRISING RASPBERRY KETONE

(71) Applicant: KOIVUBIOTECH OY, Helsinki (FI)

(72) Inventor: Sari SALMINEN, Helsinki (FI)

(21) Appl. No.: 17/796,251

(22) PCT Filed: Jan. 28, 2021
(86) PCT No.: PCT/FI2021/050054

§ 371 Date: Jul. 28, 2022

(30) Foreign Application Priority Data
Jan. 29, 2020 (FI) 20205086

(51) Int. Cl. A61K 8/35; A61K 8/9789; A61K 47/10; A61K 47/26; A61Q 19/00; A23L 33/105; A23L 33/125

(52) U.S. Cl. CPC A61K 8/35; A61K 8/9789; A61K 47/10; A61K 47/26; A61Q 19/00; A23L 33/105; A23L 33/125; A61K 2800/10

(57) ABSTRACT

The present invention describes a composition comprising raspberry ketone and at least one monosaccharide and optionally at least one phenolic compound. The invention also describes a method for extracting with an aqueous solvent a composition comprising raspberry ketone from the inner bark of birch. The extract thus obtained can optionally also comprise monosaccharides and phenolic compounds.

### FIELD OF THE INVENTION

The current invention relates to a composition comprising natural components, which are extractable from birch in general. More specific the current invention relates to a composition comprising raspberry ketone and monosaccharides, which are obtainable by extraction of the bark of birch trees. The current invention also relates to a method of extracting the bark of birch to obtain a composition comprising raspberry ketone and monosaccharides.

### BACKGROUND OF THE INVENTION

Raspberry ketone is a phenolic compound that occurs naturally in several berries such as cranberries, blackberries and raspberries. Raspberry ketone has a fruity, berry-like odour and is therefore regularly used in cosmetics, such as soaps and perfumes, but also in foodstuff as an additive. Several health-related properties have also been linked to raspberry ketone. For example, raspberry ketone has been used in weight-loss preparations and in skin and hair-loss preparations.

Raspberry ketone, or 4-(4-hydroxyphenyl)butan-2-one, can be extracted from raspberries, but since the abundance in the berries is very low, it is prepared industrially either through a chemical synthetic route or by micro-organism fermentation. Publication GB 2416769 describe a fermentation process, wherein a host cell comprising a benzalacetone synthase (BAS) polypeptide sequence and a 4-coumarate:CoA ligase (4CL) sequence, is capable of producing e.g. raspberry ketone from a p-coumaric acid precursor. Alternatively, raspberry ketone can be chemically synthesized from 4-hydroxybenzaldehyde.

Especially in foodstuff and food supplements but also in cosmetic uses it is desirable to use natural products and preparations compared to synthetic preparations. The consumer typically favours natural products over synthetic preparations. There is therefore a need for new natural sources and methods for obtaining raspberry ketones.

### BRIEF DESCRIPTION OF THE INVENTION

An object of the present invention is to provide a new natural source for raspberry ketone. The objects of the invention are achieved by a method, a composition and an extract as they are characterized by what is stated in the independent claims. The preferred embodiments of the invention are disclosed in the dependent claims. The invention is based on the notion that raspberry ketone can be obtained as a composition from the birch tree, especially by extraction of the inner bark of birch tree.

The present invention therefore provides a composition comprising
raspberry ketone, and
one or more monosaccharide(s).

Further, the present invention provides a method to obtain a composition comprising raspberry ketone, wherein the method comprises:
obtaining a biomass of inner bark of birch,
extracting the biomass with an aqueous solvent, and
collecting from the aqueous solvent a composition comprising raspberry ketone.

Still further, the present invention provides an extract of inner bark of birch tree, wherein the extract comprises raspberry ketone.

Embodiments of the invention are presented in the dependent claims.

An advantage of the invention is that it provides a new source, namely bark of tree, for raspberry ketone, especially the source is inner bark of birch tree. Previous sources for natural extracts of raspberry ketone include berries and some fruits. This has led to a scarcity of natural extracts containing raspberry ketone due to low amount of the ketone in the berries and the availability of berries.

In addition, an advantage of the present invention is that a composition comprising raspberry ketone and naturally occurring monosaccharides with various beneficial properties is acquired.

The present invention provides an easy, versatile and economically profitable method of obtaining natural composition comprising raspberry ketone.

The method comprises using birch tree and especially the inner bark of birch tree as the source and extracting a composition according to the invention from the inner bark of birch tree. Compared to berries and previous sources of raspberry ketone, inner bark of birch is cheap, present in abundance and easy to process.

One advantage of the present invention is that it provides use for left-over material produced in forestry. Today there is a need to for a more complete utilisation of by-products from forestry and using trees for traditional purposes. There is a need for green biotech and utilisation of natural products for the productions of chemical compounds and composition. In circular economy it is important to minimise waste production and maximise utilisation of various by-products to produce valuable products.

The current invention provides a novel natural and renewable source for compositions comprising raspberry ketone. The invention thereby reduces the need for synthetic production of raspberry ketone and provides an environmentally friendly, sustainable and biological method for obtaining natural composition comprising raspberry ketone.

### DETAILED DESCRIPTION OF THE INVENTION

In one aspect, the invention provides a composition comprising raspberry ketone and one or more monosaccharides. Raspberry ketone is a phenolic compound that occurs naturally in the berry raspberry and is the primary aroma compound of raspberries. The systematic organic chemical name of raspberry ketone is 4-(4-hydroxyphenyl)butan-2-one or p-hydroxybenzyl acetone. The chemical structure of raspberry ketone is presented in formula (I)

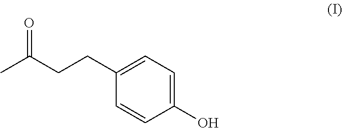

Monosaccharides are the basic units of carbohydrates and typically constitute three, four, five, six or seven carbon atoms. In one aspect of the current invention the monosaccharides of the composition are pentoses (5 carbon) and/or hexoses (6 carbon). The monosaccharides of the composition can be naturally occurring monosaccharides that are extractable from the bark of birch tree, preferably the inner bark of birch tree.

In one aspect of the invention it is provided an extract of bark of birch tree, preferably the inner bark of birch tree. It has surprisingly been found that the inner bark of birch tree contains raspberry ketone in an extractable form. The invention therefor provides a composition obtainable from the inner bark of birch and an extract from inner bark of birch tree, wherein both the composition and the extract comprise raspberry ketone and one or more monosaccharides, and the monosaccharides are preferably natural monosaccharides obtainable from or by a birch bark extraction.

In one aspect of the invention the monosaccharide or monosaccharides of the composition and/or extract is/are selected from the group of arabinose, pyranoses, furanoses, psicose, rhamnose, mannose, fucose and any combination thereof. Preferably the monosaccharide(s) is/are selected from the group of D-psicose, D-mannose, L-fucose, L-rhamnose and any combination thereof.

The composition of the current invention comprising a raspberry ketone and one or more monosaccharide provides a novel natural product that can be used in several applications, including, but not limited to, as an ingredient in foodstuff or food supplements, enhancing the aroma, flavour or scent of various products and in cosmetical used. The combination of raspberry ketone and monosaccharides, especially naturally occurring monosaccharides, provides beneficial properties such as a composition with combined flavouring or aroma and sweetener properties. Many of the naturally occurring monosaccharides, extractable from inner bark of birch tree together with raspberry ketone, such as psicose, rhamnose, fucose, mannose and arabinose, function as natural nutritive and non-nutritive sweeteners and together with raspberry ketone they function as a ready to use composition in foodstuff.

Many of the monosaccharides naturally present in bark of birch are useful and regularly used also in cosmetic applications. Raspberry ketone also has beneficial properties, which can be utilised in cosmetic application. It is therefore beneficial to provide a ready to use composition comprising raspberry ketone and at least one monosaccharide.

In one embodiment of the invention, the composition and extract according to the invention further comprise phenolic compounds. The phenolic compounds of the composition or extract can be natural phenolic compounds obtainable from a birch tree bark extract, especially an inner bark extract of birch tree. Examples of extractable phenolic compounds present in inner bark of birch tree besides raspberry ketone include, but are not limited to phenolic acids like vanillic acid and syringic acid; phenolic aldehydes like vanillin, coniferyl aldehyde, syringaldehyde, sinapaldehyde and phenolic alcohols like rhododendrol, eugenol, mequinol, guaiacol, p-coumaryl alcohol, coniferyl alcohol and sinapyl alcohol. In one aspect of the invention the composition and extract according to the invention further comprises a phenolic compound selected from the group of vanillic acid, syringic acid, coniferyl aldehyde, syringaldehyde, sinapaldehyde, rhododendrol, vanillin, eugenol, mequinol, guaiacol, p-coumaryl alcohol, coniferyl alcohol, sinapyl alcohol and any combination thereof. Preferably the composition and extract according to the invention further comprises rhododendrol, vanillin or any combination thereof.

The phenolic compounds in the composition and extract of the invention have beneficial bioactive properties. Rhododendrol or vanillin and other phenolic compounds, can function as tyrosinase inhibitors. Tyrosinase catalyses the break down of other phenolic compounds, found in fruits and vegetables, to quinones, which have an unwanted taste and colour. Tyrosinase inhibitors are therefore wanted in foodstuff. Many phenolic compounds are also natural antioxidants.

The composition according to the invention has surprisingly been found to contain anti-microbial activity, especially against the growth of bacteria Bacillus cereus. The bacteria B. cereus are Gram-positive bacteria commonly found in soil and food and are known to cause foodborne illnesses. The anti-microbial activity of the composition is more profound when only water is used as the aqueous solvent in the extraction. The anti-microbial activity is preserved in the composition and/or extract according to the invention when then aqueous extraction of the biomass is performed in a temperature from 30° C. to 200° C., preferably from 40° C. to 200° C.

According to another aspect the invention provides a method to obtain a composition or extract comprising raspberry ketone, wherein the method includes extracting a biomass comprising inner bark of birch with an aqueous solvent and collecting from the aqueous solvent a composition or extract comprising raspberry ketone.

With birch or birch tree is here meant any tree of the genus Betula, such as Betula pendula. Birch is a hard wood from which plywood and veneer is produced, which can be used for example in furniture or other indoor uses. The bark of birch can be separated into inner bark and outer bark.

In one aspect of the method the collecting of the composition comprising a raspberry ketone from the aqueous solvent is performed by drying the aqueous solvent to obtain a dry residue. The drying can be performed by any conventional drying method such as, but not limited to, evaporation, spray-drying, freeze drying or a combination thereof.

In another aspect of the method the collecting of the composition comprising raspberry ketone from the aqueous solvent is performed by concentrating the aqueous solvent with a suitable method. Concentration of the aqueous solution can be performed by evaporation with or without heating of the aqueous solution. When the aqueous solution is concentrated to obtain the composition the raspberry ketone and at least one monosaccharide and optionally at least one phenolic compound is dissolved in the aqueous solution. The aqueous solution can be used as such e.g. as an aroma-enhancer or sweetener in various beverages or other liquid or near liquid foodstuff, such as smoothies, yoghurt and other dairy products.

In one embodiment of the invention the aqueous solution is purified after extraction but before drying or concentration. The purification of the aqueous solution can be performed by a liquid-liquid extraction, solid phase extraction, column chromatography or the like. In a preferred embodiment the aqueous solution is dried or concentrated directly after the extraction step without additional purification steps.

In one embodiment of the invention one extraction of the biomass comprising inner bark of birch tree is performed to obtain the composition comprising raspberry ketone. It can be sufficient with only one extraction to obtain the desired or pre-determined composition comprising raspberry ketone. Performing only one extraction is beneficial since it makes the process simple, fast and easier to scale-up. The composition obtained from one extraction can be further purified with for example a liquid-liquid extraction using a non-polar solvent to extract lipophilic components from the aqueous solution. Alternatively, a chromatographic separation can be used to further purify the aqueous solution from one extraction.

In one embodiment of the invention more than one extraction are performed. When more than one extraction is used preferably the extraction conditions, such as temperatures are different in the various extractions. By varying the extraction conditions such as temperature and solvent-mixture it is possible to obtain solutions with different compositions of monosaccharides and/or phenolic compounds. Thereby, it is possible to reach a desired or pre-determined composition by performing a series of more than one extraction without the need excessive purification of the aqueous solvent. The aqueous solvents from the various extracts are combined and dried or concentrated to obtain the final composition or extract comprising raspberry ketone.

The composition or extract obtained from the method according to the present invention is either in the form of a dried residue or an aqueous solution (concentrate). If the composition is in the form of an aqueous solution the volume of the aqueous solvent used in the extraction is concentrated to obtain the final aqueous solution. A dried composition can also be re-dissolved to obtain an aqueous solution (concentrate).

In one aspect of the method the extraction of the biomass comprising inner bark of birch is performed in a temperature from 30° C. to 200° C., preferably from 60° C. to 180° C., and more preferably from 90° C. to 150° C. The extraction is performed under pressure to secure sufficient yield of raspberry ketone in the extract. In one embodiment the extraction is performed using a so called pressurised hot water extraction (PHWE). PHWE is a form of pressurised liquid extraction in which hot water or an aqueous solvent, such as over 50° C. is used as the extraction medium and the extraction is performed under high pressure. The high pressure applied decreases the polarity of the water as a solvent, and the water becomes more non-polar making extraction of more hydrophobic compounds possible, compared to extraction without pressurising. The pressure depends on the temperature used in the extraction process. The most important parameter in PHWE, beside the dielectric constant (e), which describe the polarity of the solvent, is temperature. The pressure is selected to keep the water at near critical state. PHWE is well documented in the literature and the person skilled in the art is able to apply PHWE to biomass either in batch or continuous mode. PHWE can also be called superheated water extraction and is related to other extraction techniques such as supercritical water extraction (SWE) and pressurized liquid extraction (PLE). The parameters and process of PHWE is described in detail for example in M. Plaza and C. Turner [Trends in Analytical Chemistry, 2015, 71, 39-54].

The extraction can also be performed using accelerated solvent extraction (ASE). The parameters and process of ASE is described in detail for example in Richter B. E. et al. [Anal. Chem. 1996, 68, 1033-1039].

Raspberry ketone is readily extracted to the aqueous solvent in almost any temperature such as over 30° C. However, with using higher temperature in the extraction more monosaccharides are extracted. Thereby, the amounts and species of monosaccharides in the composition or extract can be controlled by using different temperatures.

In one embodiment a predetermined temperature is used for the extraction. The extraction can be performed one time or more than one time in the same temperature. If more than one extraction is used the extracts can be combined. In another embodiment an extraction protocol is used where the temperature is increased stepwise during the extraction. In this extraction mode the extraction is started at a lower temperature, such as from 30° C. to 140° C. The temperature is then increased one or more times to reach a final extraction temperature, such as from 160° C. to 200° C. With this extraction process all possible monosaccharides and phenolic compounds can be extracted in a single process using an increasing temperature process, without the need for several extractions that need to be combined.

Generally, monosaccharides are extracted in all temperatures. Some monosaccharides extract more easily compared to others that need higher temperatures to be extracted. Thereby, the distribution of the various monosaccharides (species) can to some extent be controlled by using certain temperatures.

By varying the temperature of the extraction process, it is also possible to vary the amount and which phenolic compounds are extracted. Thereby, the temperature of the aqueous solvent during the extraction is one of the most important factors for controlling the monosaccharides and phenolic compounds of the composition and extract obtained from the extraction process. By carefully selecting the temperature depending on the wanted mixture of monosaccharides and/or phenolic compounds in the composition or extract obtained from the extraction process, less purification or post-extraction processes are required to obtain the desired composition.

The person skilled in the art is well familiar with various extraction processes and the current invention is not restricted to a specific extraction process. The extraction vessel or more precisely the water used for extraction need to be kept under pressure to keep it in liquid form especially when higher temperatures are used. The person skilled in the art is well familiar with various methods and equipment, which can be used for performing a water extraction in high temperatures and pressure.

The person skilled in the art is also capable of controlling and varying the extraction time. The current extraction process is not restricted to any specific extraction time, instead the person skilled in the art can vary and control the extraction time by analysing the obtained composition.

In one embodiment of the invention, the inner bark of birch tree is separated from the outer bark and the inner bark is crushed and/or grinded before the extraction. The biomass comprising inner bark of birch is preferably produced by obtaining birch bark, separating the inner bark from the outer bark and using only the separated inner bark as the biomass for extraction. It is preferred to use mainly just the inner bark of birch to ensure good yield and predictable quality of the biomass.

In one embodiment of the invention the aqueous solvent used for extracting the birch bark biomass is water or a mixture of water and an organic solvent selected from ethanol, ethyl acetate, methanol, tetrahydrofuran (THF), acetone and any mixture thereof. Preferably the aqueous solvent is water. Raspberry ketone is extracted from the bark of birch using only water as the extraction solvent. Depending on the desired mixture of other components, especially phenolic compounds, that are desired in the composition or extraction it can be useful to add some organic solvent to the aqueous solvent to alter the polarity of the aqueous solvent.

In one embodiment of the invention the aqueous extract can undergo a post-extraction process to purify and/or separate the various monosaccharides and phenolic compounds from each other. In one embodiment the aqueous extract is extracted using an organic solvent, such as dichloromethane, acetone, hexane, heptane, toluene, ethyl acetate, MTBE, THF or diethyl ether. With a non-polar solvent, the phenolic compounds, including rhododendrol and/or vanillin together with raspberry ketone is transferred to the organic phase. The monosaccharides remain in the aqueous phase. In the organic phase, if necessary rhododendrol can easily be oxidised to raspberry ketone. Oxidation of rhododendrol can be achieved chemically or using an enzyme, such as alcohol dehydrogenase. Oxidation of rhododendrol therefore increases the overall yield of raspberry ketone. The monosaccharides of the aqueous phase can then be combined with the raspberry ketone comprising composition of the organic phase to obtain the final composition.

Another aspect of the current invention is to provide an extract of inner bark of birch tree, wherein the extract comprised raspberry ketone. In one embodiment the extract is a water extract. In one embodiment the extract further comprised monosaccharides and/or phenolic compounds. The preferred monosaccharides and phenolic compounds of the extract are the same preferred monosaccharides and phenolic compounds of the composition according to the invention.

Still another aspect of the invention is to provide a dry extract residue obtainable from a water extract of inner bark of birch, wherein the dry extract residue comprises raspberry ketone.

The composition and/or extract of the current invention can be used in any application where raspberry ketone is typically used. The composition can be used as a raspberry tasting sweetener, as a food additive to add raspberry taste, aroma or scent to any foodstuff, such as smoothies, milk, yoghurt, ice cream and other dairy products, non-alcoholic and alcoholic beverages, oat products and sweet bakery products. The composition and/or extract of the current invention can also be used in cosmetical products providing raspberry aroma. Since the preparation of the composition and extract of the invention require only limited preparation at relatively mild conditions and no toxic solvents need to be used, all the beneficial properties of the natural products are preserved.

### EXAMPLES

### Example 1

Sample Preparation and Extraction

Freeze-dried bark samples from Betula pendula birch were separated into the inner and the outer layer of birch bark. After that inner bark was grinded into a particle size of <0.5 mm with a rotor mill.

Extraction experiments were performed with Dionex-350 ASE (Accelerated Solvent Extractor) system (Dionex Co, Sunnyville, Calif., USA). A sample of 0.5 g of dried homogenized bark was weighed into the stainless steel 22 mL ASE extraction cell with a cellulose fiber filter of 30 mm i.d. (Dionex Co, Sweden). ASE extractions were made by using the following program: Heating time 5 min at 90° C., 7 min at 140° C., and 9 min at 180° C., pressure at 1500 psi, 15 min static time, and 3 cycles of static extraction. At the end of a cycle, cell was rinsed with a solvent of 25% of cell volume and purged for 120 s with nitrogen to dry all the tubing. This was extraction with water were made at 90, 140, and 180° C. temperatures.

After extraction, if necessary, the extract is subjected to a purification/filtration step e.g. with a membrane. In order to have a liquid extract of comprising composition of Raspberry ketone-monosugars the purified/filtrated liquid extract was partially concentrated in a rotary vacuum evaporator. In order to have a solid composition the purified/filtrated liquid extract was lyophilized into solid form.

Sample Analysis

All the extracts (10 mL) were concentrated to 2 mL of volume under a gentle stream of nitrogen. 1 mL of the extract for the analysis of non-volatile compounds was silylated with a mixture of BSTFA (504), TMCS (50 μL) and pyridine (100 μL) at 70° C. for 20 minutes. After silylation, the dry residue was dissolved in 1 mL of dichloromethane. Second non-silylated sample aliquot was evaporated to dryness under nitrogen flow and reconstituted with 1 mL of dichloromethane. 1 mL of aqueous sample was put into a freeze dryer for 24 hours. Then a freeze-dried Betula pendula sample was silylated and reconstituted with DCM. Finally, sample aliquots were filtered through a polytetrafluoroethylene (PTFE) membrane filter (pore size of 0.45 μm) before GC×GC analysis.

Blank samples were made in order to monitor potential contamination (extraction, silylation, GC×GC-TOFMS-analysis) through the entire process and triplicate samples were used to increase reliability of analyses.

Comprehensive two-dimensional gas chromatography-time-of-flight mass spectrometry analysis were carried out on an Agilent© 7890 Gas Chromatograph (Santa Clara, USA) equipped with a split/splitless injector and coupled to a LECO Pegasus® 4D TOFMS system (LECO, St Joseph, Mich., USA). Agilent GC was equipped with a secondary oven and a liquid N2 dual stage-jet thermal modulator.

The 1st dimension column was a non-polar BGB-5MS (30 m×0.25 mm i.d., 0.25 μm film thickness) and the 2nd dimension column a semi-polar DB-17 (1 m×0.1 mm i.d., 0.10 μm film thickness) installed in the secondary oven. The columns were connected with a glass press-fit-connector (Restek, Bellefonte, Pa., USA). A 4 m×0.32 mm i.d. diphenyl tetramethyl disilazane (DPTMDS) deactivated retention gap was connected to the first-dimension column.

A volume of 14 extract was injected in splitless mode at 280° C. Helium was used as the carrier gas at a constant flow rate of 1.3 mL/min. The temperature of the first-dimension column was programmed as follows: 30° C. for 2 min, at 10° C./min to 280° C. and held for 10 minutes. The second-dimension column was programmed as follows: 35° C. for 2 min, at 10° C./min to 285° C. and held for 10 minutes. The total GC×GC run time was 47 minutes.

The cryogenic cooling modulator was placed between the two columns. The modulator temperature offset, relative to the secondary oven temperature, was 15° C. Modulation period was 6 s with a 0.60 s hot pulse time and a 2.40 s cooling time between stages. The transfer line between GC×GC and the TOF-MS system was maintained at 290° C. The ion source temperature was 200° C. with an electron impact (EI) energy of 70 eV. Data were collected, after a solvent delay of 510 seconds, in the mass range of 50-700 u with an acquisition rate of 50 spectra/s, using a detector voltage of 1800 V.

The accurate mass measurements were confirmed and performed by an Agilent HP model 6890N gas chromatograph (Agilent Technologies, Pittsburgh, Pa., USA) coupled to an HP 5973A quadrupole mass spectrometer interfaced with a time-of-flight mass spectrometry.

The analysis results for the various monosaccharides and phenolic compounds can be seen in table 1 and table 2 respectively.

TABLE 1
Combined results of identified carbohydrates (analysed with GC×GC-TOF-MS and confirmed with
GC-QTOF-MS) extracted at three different temperatures. ASE denotes Accelerated Solvent Extractor

| | ASE | ASE | ASE |
|---|---|---|---|
| Compound | 90° C. | 140° C. | 180° C. |
| Sugars | | | |
| 1,5-Anhydrohexitol, 4TMS derivative | | x | x |
| 2-à-Mannobiose, octakis(trimethylsilyl) ether (isomer 1) | | x | x |
| D-Mannose | | | x |
| á-D-Lactose, (isomer 2), 8TMS derivative | | | x |
| D-Psicose, 5TMS derivative | x | x | |
| D-(+)-Turanose, octakis(trimethylsilyl) ether | x | x | x |
| L-Fucose, 4TMS derivative | | | x |
| L-Rhamnose, 4TMS derivative | | | x |
| Melibiose, octakis(trimethylsilyl)-, disaccharide | x | x | |
| Furanoses | | | |
| 2-Deoxypentofuranose | x | x | |
| D-(−)- Erythrofuranose, tris(trimethylsilyl) ether (isomer 2) | | x | |
| D-(−)-Fructofuranose, pentakis(trimethylsilyl) ether (isomer 1) | | x | |
| D-(−)-Lyxofuranose, tetrakis(trimethylsilyl) ether | | | x |
| D-Psicofuranose, pentakis(trimethylsilyl) ether (isomer 2) | | | |
| D-(−)-Ribofuranose, tetrakis(trimethylsilyl) ether (isomer 1) | x | x | x |
| D-(−)-Ribofuranose, tetrakis(trimethylsilyl) ether (isomer 2) | x | | x |
| D-(−)-Tagatofuranose, pentakis(trimethylsilyl) ether (isomer 1) | x | x | x |
| Methyl à-Arabinofuranoside, 3TMS derivative | | | x |
| Methyl à-D-glucofuranoside, 4TMS derivative | x | | |
| Pyranoses | | | |
| à-D-(−)-Lyxopyranose, 4TMS derivative | x | x | |
| á-D-(−)-Ribopyranose, 4TMS derivative | | x | |
| á-D-(+)-Mannopyranose, 5TMS derivative | x | x | |
| á-D-(+)-Talopyranose, 5TMS derivative | x | x | |
| á-D-(+)-Xylopyranose, 4TMS derivative | x | x | x |
| à-D-Galactopyranose, 1,2,3-tris-O-(trimethylsilyl)-, cyclic | | x | x |
| methylboronate | | | |
| á-D-Galactopyranoside, methyl 2,3-bis-O-(trimethylsilyl)-, cyclic methylboronate | | x | |
| à-D-Glucopyranoside, methyl 2-(acetylamino)-2-deoxy-3-O-(trimethylsilyl)-, | | x | x |
| cyclic methylboronate | | | |
| à-d-Glucopyranoside, methyl 2,3,4-tris-O-(trimethylsilyl)-, hexadecanoate | | | x |
| á-D-Glucopyranose, 1,6-anhydro- | x | | |
| á-D-Glucopyranose, 5TMS derivative | x | | x |
| à-D-Mannopyranose, 5TMS derivative | | x | |
| á-D(−)-Lyxopyranose, 4TMS derivative | | | x |
| à-D-Xylopyranose, 4TMS derivative | | | x |
| D-(+)-Galactopyranose, 5TMS derivative (isomer 2) | | x | x |
| D-Arabinopyranose, 4TMS derivative (isomer 2) | x | x | |
| D-Erythro-Pentopyranose, 2-deoxy-1,3,4-tris-O-(trimethylsilyl)- | x | | |
| D-Psicopyranose | x | | |
| DL-Arabinopyranose, 4TMS derivative | | | x |

TABLE 2
Combined results of identified phenolic compounds (analysed with GC×GC-TOF-MS and confirmed with GC-QTOF-MS)
extracted at three different temperatures. ASE denotes Accelerated Solvent Extractor

| | | ASE | ASE | ASE |
|---|---|---|---|---|
| Compound | Synonym | 90° C. | 140° C. | 180° C. |
| (E)-; Phenol, 2,6-dimethoxy-4-(1E)-1-propenyl- | trans-4-Propenylsyringol | | x | x |
| 1-(4-Hydroxybenzylidene)acetone | p-Hydroxybenzalacetone | x | x | x |
| 1,4-benzenediol, 2,5-dimethoxy- | 2,5-dimethoxyhydroquinone | | x | x |
| 1-(4-Hydroxy-3-methoxyphenyl)butan-1-one | Butyrovanillone | | x | x |
| 2-Butanone, 4-(4-hydroxyphenyl)- | Raspberry ketone | x | x | x |
| 2-Methoxy-4-vinylphenol | p-Vinylguaiacol | | x | x |
| 2-Propanone, 1-(4-hydroxy-3-methoxyphenyl)- | Guaiacylacetone | | | x |
| 2-Propanone, 1-hydroxy-3-(4-hydroxy-3-methoxyphenyl)- | | | | x |
| 2,3-Dihydroxybenzaldehyde | | | | x |
| 3-(4-hydroxy-3-methoxyphenyl)prop-2-enal | Coniferyl aldehyde | x | x | x |
| 3-(4-Hydroxyphenyl)propanal | 4-Hydroxydihydrocinnamaldehyde | x | x | x |
| 3,4,5-Trimethoxyphenol | | | | x |
| 3,5-Dimethoxy-4-hydroxycinnamaldehyde | Sinapaldehyde | x | x | x |
| 3,5-Dimethoxy-4-hydroxyphenethylamine | | | | x |
| 4-Hydroxybenzamide | | | | x |
| 4-((1E)-3-Hydroxy-1-propenyl)-2-methoxyphenol | Coniferyl alcohol | | x | x |
| 4-Allyl-2-methoxyphenol | Eugenol | | x | x |
| 4-Hydroxy-3-methoxybenzaldehyde | Vanillin | x | x | x |
| 4-Methoxyphenol | Mequinol | | x | x |
| 4-(1-Hydroxyallyl)-2-methoxyphenol | 1′-Hydroxyeugenol | | | x |
| 5-(3-Hydroxypropyl)-2,3-dimethoxyphenol | Benzenepropanol, 3-hydroxy-4,5-dimethoxy- | | x | x |
| Acetophenone, 4-hydroxy- | Piceol | | x | x |
| Benzaldehyde, 4-hydroxy- | p-Formylphenol | | x | x |
| Benzaldehyde, 4-hydroxy-3,5-dimethoxy- | Syringaldehyde | x | x | x |
| Benzene-1,3-diol | Resorcinol | | x | x |
| Benzene-1,4-diol | Hydroquinone | | x | x |
| Benzenepropanol, 4-hydroxy-à-methyl-, (R)- | Rhododendrol | x | x | x |
| Benzoic acid, 3-methoxy-4-[(trimethylsilyl)oxy]- | Vanillic acid | | x | x |
| Ethanone, 1-(4-hydroxy-3,5-dimethoxyphenyl)- | Acetosyringone | | x | x |
| Phenol, 2,6-dimethoxy- | Syringol | | | x |
| Phenol, 2-methoxy- | Guaiacol | | x | x |
| Phenol, 2-methoxy-3-(2-propenyl)- | 3-Allylguaiacol | | x | x |
| Phenol, 2-methoxy-4-(1-propenyl)- | Isoeugenol | x | x | x |
| Phenol, 2-methoxy-4-(2-propenyl)-, acetate | Acetyleugenol | | x | x |
| Phenol, 2-methoxy-4-propyl- | Cerulignol | | x | x |
| Phenol, 2-propyl- | | | | x |
| Phenol, 3-ethyl- | | | x | x |
| Phenol, 4-[(1E)-3-hydroxy-1-propenyl]-2,6-dimethoxy- | Sinapyl alcohol | | | x |
| Phenol, 4-hexyl- | | | | x |

### Example 2

Sample preparation was performed as in Example 1. The extraction was performed by raising the temperature during the extraction process. The extraction process was as follows: the first fraction can be collected at 140° C. for 30 minutes and the second fraction after raising the temperature to 180° C. for 40 minutes.

The combined water fractions were analysed as in Example 1. Results show that the same monosaccharides and phenolic compounds as in Example 1 and listed in Table 1 and Table 2 could be extracted using an extraction process where the temperature is raised stepwise during the extraction process.

It will be obvious to a person skilled in the art that, as the technology advances, the inventive concept can be implemented in various ways. The invention and its embodiments are not limited to the examples described above but may vary within the scope of the claims.

1. Composition comprising
raspberry ketone, and
one or more monosaccharide(s).

2. Composition according to claim 1, wherein the monosaccharides are natural monosaccharides obtainable from a birch tree bark extraction.

3. The composition according to claim 1, wherein the monosaccharides are selected from the group of arabinose, pyranoses, furanoses, psicose, rhamnose, mannose, fucose and any combination thereof.

4. The composition according to claim 1, wherein the monosaccharides are selected from the group of D-psicose, D-mannose, L-fucose, L-rhamnose and any combination thereof.

5. The composition according to claim 1, wherein the composition further comprises phenolic compounds.

6. The composition according to claim 1, wherein the composition further comprises phenolic compounds and the phenolic compounds are natural phenolic compounds obtainable from a birch tree bark extraction.

7. The composition according to claim 1, wherein the composition further comprise phenolic compounds and the phenolic compounds are selected from vanillic acid, syringic acid, coniferyl aldehyde, syringaldehyde, sinapaldehyde, rhododendrol, vanillin, eugenol, mequinol, guaiacol, p-coumaryl alcohol, coniferyl alcohol, sinapyl alcohol and any combination thereof, preferably selected from vanillic acid, vanillin and any combination thereof.

8. A method to obtain a composition comprising raspberry ketone, wherein the method comprises:
obtaining a biomass of inner bark of birch,
extracting the biomass with an aqueous solvent, and
collecting from the aqueous solvent a composition comprising raspberry ketone.

9. The method according to claim 8, wherein the extraction is performed as a pressurised hot water extraction (PHWE) or an accelerated solvent extraction (ASE).

10. The method according to claim 8, wherein the collecting of the composition comprising raspberry ketone is performed by drying the aqueous solvent to obtain a dry residue comprising the raspberry ketone.

11. The method according to claim 8, wherein the collecting of the composition comprising raspberry ketone is performed by concentrating the aqueous solvent.

12. The method according to claim 8, wherein the extraction of the biomass is performed in a temperature from 30° C. to 200° C., preferably from 60° C. to 180° C. and more preferably from 90° C. to 150° C.

13. The method according to claim 8, wherein the aqueous solvent is water or a mixture of water and an organic solvent selected from ethanol, ethyl acetate, methanol, tetrahydrofuran (THF), acetone and any mixture thereof; preferably the aqueous solvent is water.

14. The method according to claim 8, wherein the step of obtaining inner bark of birch comprises separating the birch inner bark from the birch outer bark and grinding the inner bark to obtain a biomass of inner bark of birch.

15. An extract of inner bark of birch tree, wherein the extract comprises raspberry ketone.

16. The extract of claim 15, wherein the extract further comprises natural monosaccharides and/or natural phenolic compounds.

17. The extract of claim 15, wherein the extract is a water extract.

18. A dry extract residue obtainable from a water extract of inner bark of birch, wherein the dry extract residue comprises raspberry ketone.
